(19) United States
(12) Patent Application Publication
(10) Pub. No.: US 2026/0124375 A1
(43) Pub. Date: May 7, 2026

(54) BIODEGRADABLE CIRCUIT AND ELECTRONIC APPARATUS

(71) Applicant: ELECTRONICS AND TELECOMMUNICATIONS RESEARCH INSTITUTE, Daejeon (KR)

(72) Inventors: Yong Suk YANG, Daejeon (KR); Kyung Hyun KIM, Daejeon (KR); Ji-Young OH, Daejeon (KR)

(21) Appl. No.: 19/296,157

(22) Filed: Aug. 11, 2025

(30) Foreign Application Priority Data
Nov. 7, 2024 (KR) 10-2024-0157088
Mar. 19, 2025 (KR) 10-2025-0035151

(51) Int. Cl. A61M 5/172; A61N 1/02; A61N 1/08; H01L 23/498; H10D 86/80

(52) U.S. Cl. CPC A61M 5/172; A61N 1/025; A61N 1/08; H10D 86/80; H10W 70/65; H10W 70/66

(57) ABSTRACT

A biodegradable circuit is disclosed. The biodegradable circuit may include a resistor. In one example, the resistor may include a substrate, a plurality of wirings disposed on an upper surface of the substrate, and a protective layer covering the plurality of wirings. For example, the upper surface of the protective layer may be divided into a first region including the center of the upper surface of the protective layer and a second region surrounding the first region and directly adjacent to an edge of the upper surface of the protective layer, and the thickness of the protective layer in the first region may be greater than the thickness of the protective layer in the second region.

### CROSS-REFERENCE TO RELATED APPLICATIONS

This application claims priority under 35 U.S.C. § 119 to Korean Patent Application No. 10-2024-0157088 filed on Nov. 7, 2024, and Korean Patent Application No. 10-2025-0035151 filed on Mar. 19, 2025 in the Korean Intellectual Property Office, the disclosures of which are incorporated by reference herein in their entireties.

### BACKGROUND

### 1. Field of the Invention

The present disclosure relates to a biodegradable circuit and an electronic apparatus.

### 2. Description of Related Art

Recently, with the advancement of electronic device technologies in the biomedical and medical fields, it has become possible to implement biodegradable electronic products that can be implanted in the human body to monitor necessary biological signals for clinical purposes and then decompose as needed when they are no longer required. This is achieved not merely by simply using materials or devices with biodegradable properties, but by utilizing such decomposition characteristics from a system-level perspective as well.

Biodegradable devices are often packaged with protective materials. The packaging material primarily determines the operational lifespan of the device by preventing direct contact between the device and bodily fluids for a specified period. These protective materials enable controlled decomposition or dissolution of the materials used in conventional implantable electronic devices within the human body and, by utilizing the packaging function, help minimize side effects such as inflammation, toxicity, or immune responses that may arise from long-term physical contact during biodegradation inside the body.

However, in order to simultaneously ensure both a stable biodegradation process and an optimized operation process for biodegradable devices and systems within the body, simply packaging the device with the protective materials has its limitations. Accordingly, ongoing research seeks to address these limitations.

### SUMMARY

The present disclosure relates to the biodegradable circuit and the electronic apparatus.

A biodegradable circuit according to an embodiment of the present disclosure comprises a resistor. The resistor comprises, a substrate, a plurality of wirings being disposed on an upper surface of the substrate, and a protective layer configured to cover an upper portion of the plurality of wirings. An upper surface of the protective layer is divided into a first region including a center of the upper surface of the protective layer, and a second region surrounding the first region and directly adjacent to an edge of the upper surface of the protective layer. A thickness of the protective layer in the first region is greater than a thickness of the protective layer in the second region.

A biodegradable circuit according to another embodiment of the present disclosure comprising a resistor. The resistor comprises a substrate, a plurality of wirings disposed on an upper surface of the substrate, a first protective layer configured to cover an upper portion of the plurality of wirings, a second protective layer disposed on an upper surface of the first protective layer. An upper surface of the resistor is divided into a first region including a center of the upper surface of the resistor and a second region surrounding the first region and directly adjacent to an edge of the upper surface of the resistor. An area of the first protective layer corresponds to an area of the upper surface of the resistor, which is equal to a sum of an area of the first region and an area of the second region. An area of the second protective layer is equal to the area of the first region.

In one embodiment, wirings included in the first region have thicker protection than wirings included in the second region.

In one embodiment, the plurality of wirings may be made of magnesium (Mg).

A biodegradable electronic device according to an embodiment of the present disclosure configured to communicate with a wireless communication device and to control an electrical stimulator and a drug dispenser, the biodegradable electronic device comprises, a memory unit configured to store a program including computer-executable instructions, a controller unit configured to execute the program, an input/output signal generator configured to generate an input voltage and an output voltage, a biodegradable reference signal generator comprising a resistor and configured to generate a reference signal, and a PWM (pulse width modulation) pulse controller configured to generate a signal for commanding turn-on or turn-off operations of the wireless communication device, the electrical stimulator, and the drug dispenser. The PWM pulse controller generates the signal for commanding the turn-on or turn-off operations of the wireless communication device, the electrical stimulator, and the drug dispenser based on at least one of the computer-executable instructions stored in the program executed by the controller unit, the input voltage and output voltage generated by the input/output signal generator, and the reference signal generated by the biodegradable reference signal generator. The reference signal is adjusted based on whether or to what extent the resistor has biodegraded.

In one embodiment, the resistor may include a biodegradable protective layer and biodegradable wirings.

### BRIEF DESCRIPTION OF THE DRAWINGS

The above and other objects and features of the present disclosure will become apparent by describing in detail embodiments thereof with reference to the accompanying drawings.

FIG. 1 illustrates a circuit diagram according to an embodiment of the present disclosure.

FIG. 2 illustrates a resistor according to an embodiment of the present disclosure.

FIG. 3, FIG. 4, and FIG. 5 illustrate cross-sectional views of a portion of a resistor according to an embodiment of the present disclosure.

FIG. 6 illustrates a resistor according to another embodiment of the present disclosure.

FIG. 7 and FIG. 8 are graphs illustrating a triangular wave signal and a duty cycle of an output voltage according to an embodiment of the present disclosure.

FIG. 9 illustrates a biodegradable electronic device according to an embodiment of the present disclosure.

FIG. 10 illustrates a biodegradable system including a biodegradable electronic device according to an embodiment of the present disclosure.

### DETAILED DESCRIPTION

Hereinafter, embodiments of the present disclosure will be clearly and elaborately described to the extent that a person skilled in the art to which the present disclosure pertains can easily carry out the present disclosure.

Terms such as “unit” and “module” used hereinafter, or functional blocks shown in the drawings, may be implemented in the form of a software configuration, a hardware configuration, or a combination thereof. In the following description, detailed descriptions of redundant components will be omitted in order to clearly explain the technical idea of the present invention.

In this document, each of phrases such as “A or B”, “at least one of A and B”, “at least one of A or B”, “A, B or C”, “at least one of A, B and C”, and “at least one of A, B, or C” may include any one of the items listed together in the corresponding phrase or all possible combinations thereof.

The present disclosure may describe embodiments including a circuit that enables a biodegradable device and system to temporarily or permanently stop operating after biodegradation proceeds to a critical state without disturbing electronic signals in the following paragraphs.

To this end, in order to simultaneously achieve a stable biodegradation process and an optimized usage process of the biodegradable device and system within the body, the present disclosure provides a structure in which the device is packaged with a protective material and interlinked with an electronic circuit, so that as the biodegradation progresses, the operation of the electronic circuit can automatically stop at an appropriate stage.

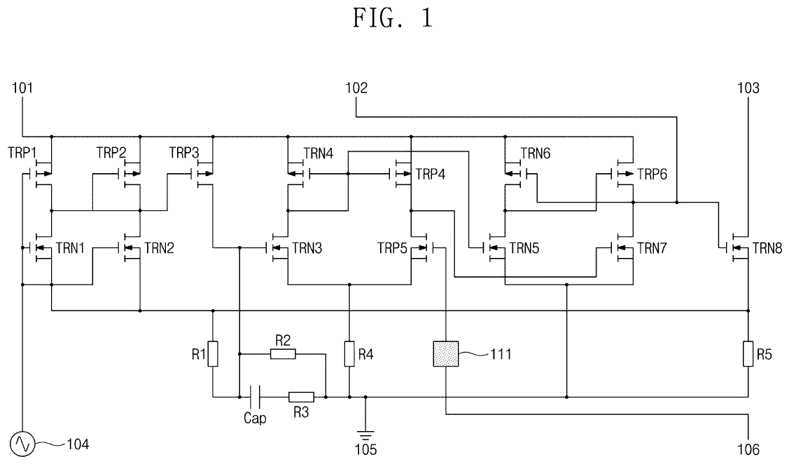

FIG. 1 illustrates a circuit diagram according to an embodiment of the present disclosure. For example, the circuit diagram of FIG. 1 may be a circuit diagram showing an example of a PWM (pulse width modulation) circuit or a modulation control circuit.

The circuit of FIG. 1 may include p-type silicon transistors TRP1, . . . , TRP6, n-type silicon transistors TRN1, . . . , TRN8, resistors R1˜R5 (for example, resistors having a size of 0.1˜100 kΩ), and a capacitor Cap (for example, a capacitor having a size of 0.1˜100 pF). A VDD voltage 101 (for example, 3.5V), an output voltage 102, a wiring for transistor testing 103, a triangular wave signal 104 (for example, a signal having a frequency of 100 Hz), a ground voltage 105 (for example, 0V), and a reference voltage 106 (for example, a reference voltage with a magnitude of 0˜3.5 V, i.e., up to 3.5V) are shown in this figure. The number of p-type and n-type silicon transistors, the number of resistors, and the number of capacitors shown in the circuit of FIG. 1 may be exemplary and are not limited to the embodiment of FIG. 1, and a person skilled in the art to which the present disclosure pertains may variously change the design.

Referring to FIG. 1, the circuit used in an embodiment of the present disclosure (for example, a pulse width modulation (PWM) control circuit) may operate by very rapidly turning on and off the power supplied to an electrical stimulation signal (output voltage 102) and the like. A DC input voltage may alternate between fully ON (for example, 3.5V) and 0, and be converted into a triangular wave signal 104 to provide a “kick” phenomenon.

In one example, a switching frequency of the triangular wave signal 104 may correspond to a duty cycle of the electrical stimulation signal (output voltage 102). When the VDD voltage 101, the ground voltage 105, and the reference voltage 106 are applied as DC input voltages, the duty cycle (pulse width, i.e., PWM modulation) of the electrical stimulation signal (output voltage 102) is obtained by comparing magnitudes of the triangular wave signal 104 and the reference voltage 106, that is, by adjusting a time ratio of an ‘On’ state, and may be changed according to the magnitude of the reference voltage 106, and accordingly, the effect of the electrical stimulation signal (output voltage 102) may also vary.

According to an embodiment of the present disclosure, a change in the magnitude of the reference voltage 106, that is, a change in a reference level, may be caused by the resistor 111. When a resistance value of the resistor 111 is the same as a resistance value of a wiring (for example, almost 0Ω), a duty cycle (or duty ratio) in the ‘On’ state may be maximized when the triangular wave signal 104 is not below or less than the reference level.

What is shown in the circuit diagram of FIG. 1 does not limit an embodiment of the present disclosure, and levels of output and input voltages of FIG. 1 may be variously changed through additional inverters or logic devices such as AND, OR, and NAND within a range modifiable by a person skilled in the art to which the present disclosure pertains.

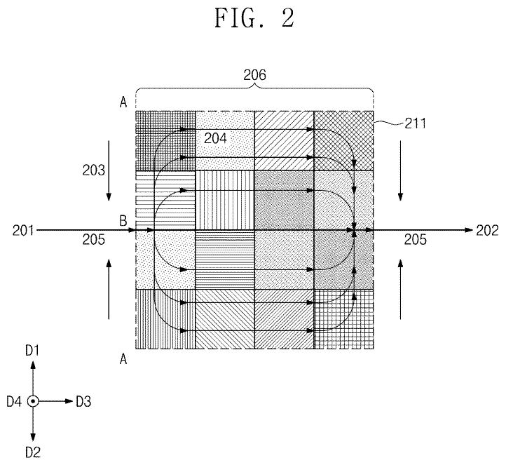

FIG. 2 illustrates a resistor according to an embodiment of the present disclosure. A resistor 211 of FIG. 2 may be the resistor 111 of FIG. 1, and may be one example of the resistor 111 of FIG. 1. FIG. 2 will be described assuming that the resistor 211 of FIG. 2 is the resistor 111 of FIG. 1, together with the reference voltage 106 of FIG. 1 and the like. In FIG. 2, a current flow 204 within the resistor 211 is shown. In FIG. 2, current may flow through the resistor 211 in a D3 direction.

Referring to FIG. 2, when the reference voltage 106 is applied to one end 201 of the wiring, since electrical conductivity of the wiring 205 (for example, the wiring may be made of magnesium (Mg)) is greater than that of other circuit components, other end 202 of the wiring may also have a voltage magnitude approximately equal to the magnitude of the reference voltage 106. In another example, if resistance exists in the central portion 206 of the wiring, a voltage drop occurs in the central portion 206 of the wiring, and the magnitude of the voltage applied to the other end 202 decreases compared to the magnitude of the reference voltage 106, which is a voltage at the one end 201 of the wiring. That is, the reference voltage 106 may be adjusted based on whether the resistor 111 has biodegraded or a degree of biodegradation.

In an embodiment of the present disclosure, as the resistor 211 biodegrades, an area of the resistor 211 may decrease in a direction 203 from position A to position B, and thus a resistance value of the resistor 211 may increase. A region of the resistor 211 located in a D1 direction with respect to the central axis of the resistor 211 parallel to the D3 direction may decompose in a D2 direction, and the region of the resistor 211 located in the D2 direction with respect to the central axis of the resistor 211 parallel to the D3 direction may decompose in the D1 direction.

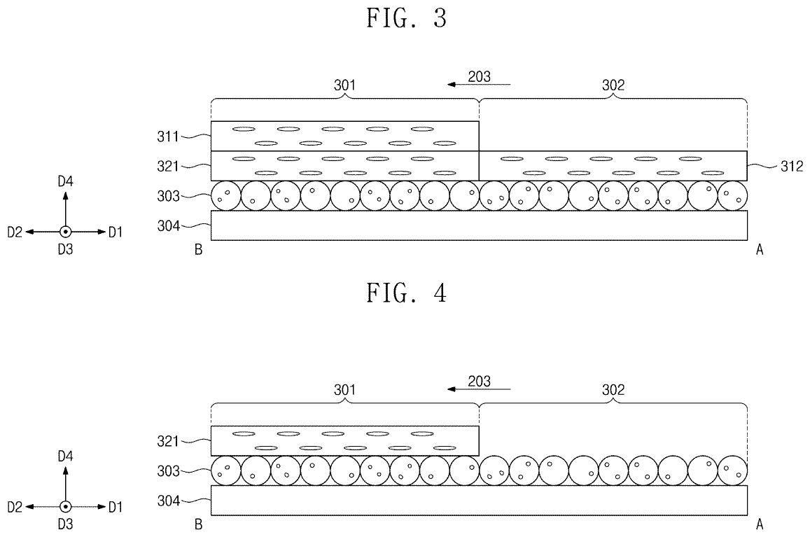

FIG. 3, FIG. 4, and FIG. 5 illustrate cross-sectional views of a portion of a resistor according to an embodiment of the present disclosure. Specifically, a cross-sectional structure of the resistor changing over time due to biodegradation from FIG. 3 to FIG. 4 and then to FIG. 5 is shown in these figures.

A resistor to be described with FIG. 3, FIG. 4, and FIG. 5 may be the resistor 211 of FIG. 2, and therefore this example may also be applied to the resistor 111 of FIG. 1. These figures are described together with FIG. 2, and for better understanding, directions corresponding to the directions of FIG. 2 are shown as D1, D2, D3, and D4. That is, a direction D4, which is a direction of an upper surface of the resistor 211 of FIG. 2, is also shown in FIG. 3, FIG. 4, and FIG. 5, and may similarly indicate a direction faced by the upper surface of the resistor 211.

Referring to FIG. 3, FIG. 4, and FIG. 5, the resistor 211 may include a substrate 304, a plurality of wirings 303, and protective layers 311, 321, 312.

The plurality of wirings 303 may be located on an upper surface of the substrate 304, that is, a surface facing the D4 direction. The protective layers 311, 321, 312 may be configured to cover an upper portion (upper part) of the plurality of wirings 303. Specifically, a first protective layer 321, 312 (although the reference numbers are distinguished, these can be viewed as a single protective layer) may be configured to cover the upper portion of the plurality of wirings 303, and a second protective layer 311 may be disposed on an upper surface of the first protective layer 321, 312 and may be configured to partially cover the first protective layer 321, 312.

The upper surface of the resistor 211 may be divided into a first region 301 including a center of the upper surface of the resistor 211, and a second region 302 surrounding the first region 301 and directly adjacent to an edge of the upper surface of the resistor 211. In FIG. 3, FIG. 4, and FIG. 5, only a portion of the resistor 211 and its cross-section are shown, so only portions of the first region 301 and the second region 302 are also shown. The entirety of the first region 301 and the second region 302 will be easier to understand by referring to FIG. 2. An area of the first protective layer 321, 312 may be equal to a sum of an area of the first region 301 and an area of the second region 302, which corresponds to an area of the upper surface of the resistor 211. An area of the second protective layer 311 may be equal to the area of the first region 301.

The protective layers 311, 321 belonging to the first region 301 closer to position B may be two layers, and the protective layer 312 belonging to the second region 302 closer to position A may be one layer. That is, wirings included in the first region 301 may have thicker protection than wirings included in the second region 302. Therefore, as exposure time of the resistor 211 to a biodegradable solution increases, the protective layer 312 belonging to the second region 302, that is, the protective layer 312 closer to position A, may first biodegrade and disappear, and wirings belonging to the second region 302 may be exposed outside the protective layer first. Therefore, the wirings 303 may sequentially dissolve in the D2 direction 203 from position A to position B. As an area (or the number) of the wirings 303 within the resistor 211 decreases, the resistance value of the resistor 211 may increase.

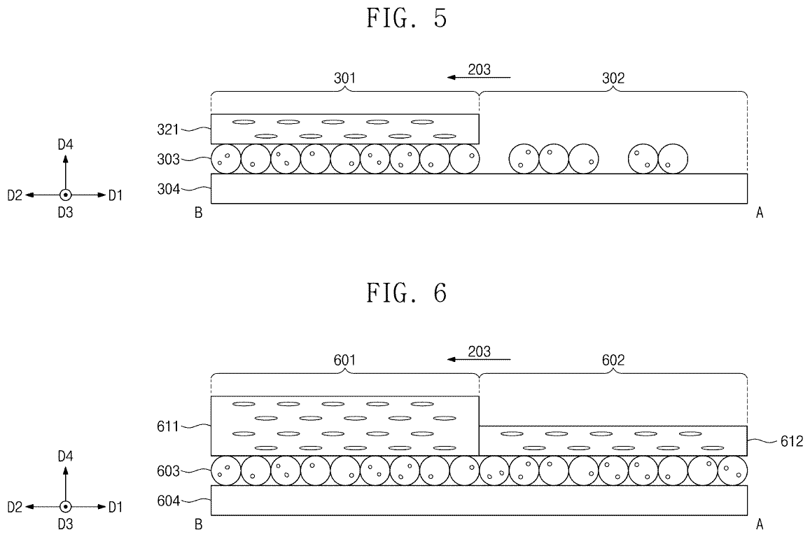

FIG. 6 illustrates a resistor according to another embodiment of the present disclosure. FIG. 6 is an embodiment characterized by a different protective layer structure from the resistor of FIG. 3, FIG. 4, and FIG. 5. FIG. 6 will also be described in a similar manner to FIG. 3, FIG. 4, and FIG. 5, and therefore, a resistor to be described with FIG. 6 may be the resistor 211 of FIG. 2, and thus this example may also be applied to the resistor 111 of FIG. 1. In FIG. 6, too, for better understanding in conjunction with FIG. 2, directions corresponding to the directions of FIG. 2 are shown as D1, D2, D3, and D4. That is, the direction D4, which is the direction of the upper surface of the resistor 211 of FIG. 2, is also shown in FIG. 6, and may similarly indicate the direction faced by the upper surface of the resistor.

In FIG. 6, the resistor 211 may include a substrate 604, a plurality of wirings 603, and protective layers 611 and 612. FIG. 6 also divides regions similarly to FIG. 3 to FIG. 5. The plurality of wirings 603 may be located on an upper surface of the substrate 604, that is, the surface facing the D4 direction. The protective layer 611, 612 (although the reference numbers are distinguished, these can be viewed as a single protective layer) may be configured to cover an upper part of the plurality of wirings 603.

In FIG. 6, a first region 601 and a second region 602 may be respectively identical to the first region 301 and the second region 302 of FIG. 3 to FIG. 5.

A distinguishing feature of the embodiment shown in FIG. 6 is that the protective layer is not formed of multiple layers. However, in order to control timing at which the wirings are exposed in each region according to biodegradation, a protective layer 611 belonging to the first region 601 closer to position B may be thicker than a protective layer 612 belonging to the second region 602 closer to position A. That is, the protective layer 612 closer to position A may be relatively thinner, while the protective layer 611 closer to position B may be relatively thicker. The thicknesses of the protective layers 611 and 612 may be determined based on factors such as a biodegradation rate of the protective layer's material. Accordingly, as exposure time of the resistor 211 to a biodegradable solution increases, the protective layer 612 belonging to the second region 602, that is, the protective layer 612 closer to position A, may biodegrade first and disappear, and wirings 603 belonging to the second region 602 may first be exposed outside the protective layer. Therefore, the wirings 603 may sequentially dissolve in the D2 direction 203 from position A to position B. As an area (or the number) of the wirings 603 within the resistor 211 decreases, the resistance value of the resistor 211 may increase.

In one example, the wirings 303, 603 of FIG. 3, FIG. 4, FIG. 5, and FIG. 6 may be made of magnesium (Mg), and the substrates 304, 604 may be made of silicon (Si).

The resistor packaged by the protective layer according to the embodiment of FIG. 6 may also be achieved by providing a manufacturing method characterized by producing it with a non-uniform thickness so as to control the resistance value.

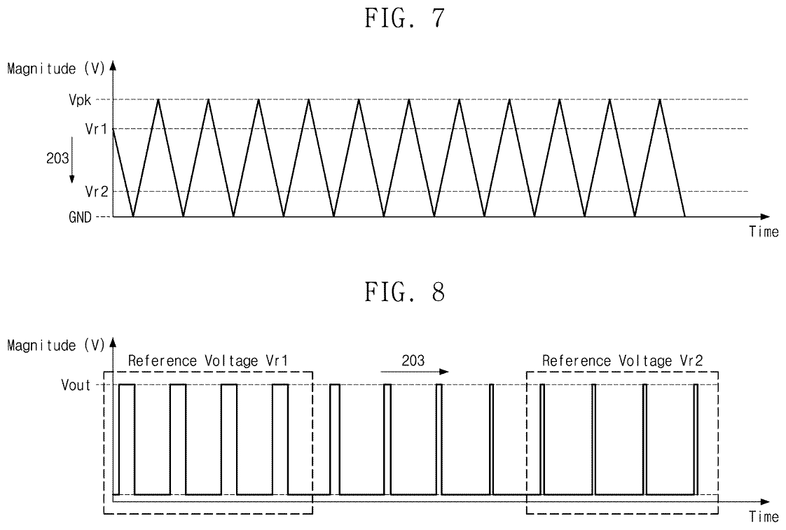

FIG. 7 and FIG. 8 are graphs illustrating a triangular wave signal and a duty cycle of an output voltage according to an embodiment of the present disclosure. FIG. 7 and FIG. 8 will be described together with the previous figures. The triangular signal of FIG. 7 may be the triangular wave signal 104 of FIG. 1, and the output voltage of FIG. 8 may be the output voltage 102 (electrical stimulation signal) of FIG. 1. In the triangular wave signal of FIG. 7, a peak value Vpk and a ground voltage GND are shown, and in the output voltage of FIG. 8, Vout, which is the maximum magnitude of the output voltage, is also shown.

In FIG. 3 to FIG. 6, it has been previously described that the wirings 303, 603 sequentially dissolve due to biodegradation over time in the D2 direction 203 from position A to position B. Accordingly, the value resistance value of the resistor 111 of FIG. 1 changes (for example, the resistance value increases), and thus the reference voltage 106 may change (for example, the reference voltage may become lower). Referring to the graph of FIG. 7, the reference voltage decreases from Vr1 to Vr2 due to the change 203 caused by biodegradation in FIG. 2 to FIG. 6. That is, the reference voltage 106 may be adjusted based on whether the resistor 111 has biodegraded or the degree of biodegradation.

Referring to FIG. 8, due to the change 203 caused by biodegradation in FIG. 2 to FIG. 6, and by the change in which the reference voltage decreases from Vr1 to Vr2, a result is obtained where the duty cycle (duty ratio, pulse width, i.e., PWM modulation) of the output voltage (output voltage 102 of FIG. 1) decreases.

In one example, in FIG. 1, if the resistor 111 is disconnected due to biodegradation, an “Off” state output of the PWM driving circuit will appear. Accordingly, the operation of the circuit may automatically stop at an appropriate stage.

Summarizing the paragraphs described together with FIG. 1, FIG. 7, and FIG. 8, the operation process of the circuit of FIG. 1 may be based on PWM driving, where an “On” state output is generated when a triangular wave voltage level of the PWM driving circuit is below or less than the reference level, and the duty cycle (pulse width, i.e., PWM modulation) of the PWM driving circuit's signal decreases due to the decreasing reference level caused by resistance generated during biodegradation, eventually resulting in an “Off” state output.

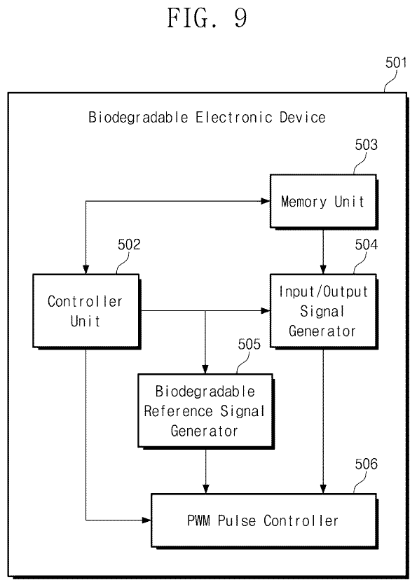

FIG. 9 illustrates a biodegradable electronic device according to an embodiment of the present disclosure. The biodegradable electronic device 501 may include a controller unit 502, a memory unit 503, an input/output signal generator 504, a biodegradable reference signal generator 505, and a PWM pulse controller 506.

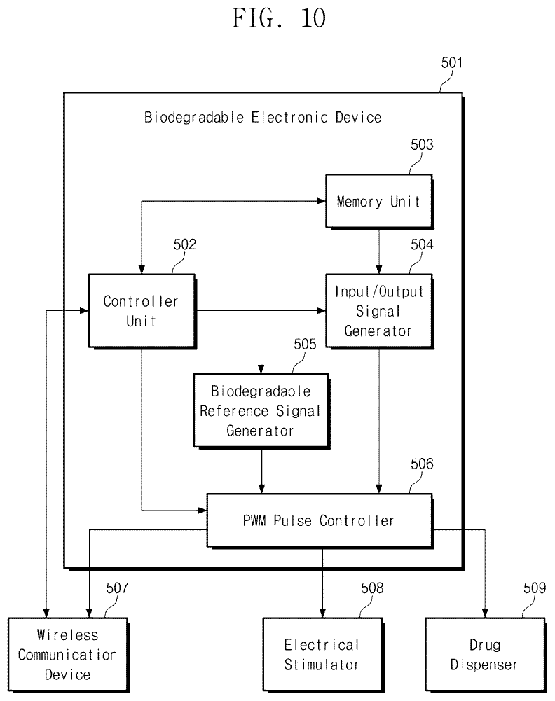

The biodegradable electronic device 501 of FIG. 9 may communicate with a wireless communication device and control an electrical stimulator and a drug dispenser. The wireless communication device, electrical stimulator, and drug dispenser will be further described in FIG. 10.

The memory unit 503 may store data and/or information, and may be configured to store a program including computer-executable instructions.

The controller unit 502 may be configured to execute a program stored in the memory unit 503.

The input/output signal generator 504 may generate input voltages (e.g., 101, 104, etc. of FIG. 1) and output voltages (e.g., 102 of FIG. 1, the signal of FIG. 8, etc.) as described in FIG. 1 to FIG. 8.

The biodegradable reference signal generator 505 may generate a reference signal / reference voltage / reference level as described in FIG. 1 to FIG. 8, and in one example, the biodegradable reference signal generator 505 may be a component such as the resistor 111 of FIG. 1 and/or the resistor 211 of FIG. 2. The reference signal generated by the biodegradable reference signal generator 505 may be adjusted based on whether the resistors 111, 211 have biodegraded or the degree of biodegradation.

The PWM pulse controller 506 may be configured to command turn-on or turn-off operation of a wireless communication device, an electrical stimulator, and/or a drug dispenser. The wireless communication device, electrical stimulator, and drug dispenser will be further described with reference to FIG. 10.

FIG. 10 illustrates a biodegradable system including a biodegradable electronic device according to an embodiment of the present disclosure. FIG. 10 will be described together with FIG. 9. The biodegradable system of FIG. 10 may include the biodegradable electronic device 501 of FIG. 9 with its components 502, 503, 504, 505, and 506, as well as a wireless communication device 507, an electrical stimulator 508, and a drug dispenser 509.

Referring to FIG. 10, the PWM pulse controller 506 may generate a signal for commanding the turn-on or turn-off operation of the wireless communication device 507, the electrical stimulator 508, and/or the drug dispenser 509 based on at least one of computer-executable instructions stored in the program executed by the controller unit 502, the input voltages and output voltages generated by the input/output signal generator 504, and the reference signal generated by the biodegradable reference signal generator 505. The signal generated by the PWM pulse controller 506 may be transmitted to the wireless communication device 507, the electrical stimulator 508, and/or the drug dispenser 509. In one example, the signal generated by the PWM pulse controller 506 may be a command to transmit instructions to the wireless communication device 507, the electrical stimulator 508, and/or the drug dispenser 509, and the wireless communication device 507, the electrical stimulator 508, and/or the drug dispenser 509 may be “turned on” or “turned off” based on the signal generated by the biodegradable electronic device 501 (generated by the PWM pulse controller 506).

For example, when the wireless communication device 507, the electrical stimulator 508, and/or the drug dispenser 509 are “turned off” , they may not perform any operation and may be in a power-off state, a deactivated state, or an idle state. When the wireless communication device 507 is “turned on” , the wireless communication device 507 may transmit a signal, information, and/or data to the controller unit 502 or receive a signal, information, and/or data from the controller unit 502. When the electrical stimulator 508 and the drug dispenser 509 are “turned on”, the electrical stimulator 508 and the drug dispenser 509 may perform their respective functions, the electrical stimulator 508 may output electrical stimulation, and the drug dispenser 509 may dispense stored drugs.

The PWM pulse control-based biodegradable electronic device according to the embodiments of the above-described paragraphs allows the output signal of the PWM circuit to be changed by biodegradation without monitoring the biodegradation process with a separate device, thereby enabling the control of a wireless communication signal, an electrical stimulation signal, and/or an electrical signal that controls drug dispensing.

As described above, the PWM pulse control-based biodegradable electronic device according to embodiments of the present disclosure may be configured by designing a structure that allows the electronic circuit to automatically stop operating at an appropriate stage as the biodegradation process proceeds, by packaging the resistor part within the circuit with a protective material having a variable thickness whose lifespan can be predicted, and by interworking it with the electronic circuit, in order to simultaneously satisfy a stable biodegradation process and an optimized usage process.

According to an embodiment of the present disclosure, the biodegradable circuit and device may be packaged with a protective layer (protective material) having a regionally variable thickness, thereby reducing the resources required to control the degradation process.

In particular, the circuit may be designed such that the operation of the electronic circuit automatically stops at an appropriate stage as the biodegradation progresses, minimizing the necessary hardware and/or software resources.

Furthermore, by forming a circuit for controlling an output voltage signal on the substrate of the biodegradable circuit and device, and by providing a protective layer with a variable thickness over the biodegradable resistor, the circuit operation can be automatically stopped as biodegradation proceeds. This makes it possible to implement a biodegradable circuit and device with broad applicability.

Embodiments according to the present disclosure are not limited to the descriptions and figures described above, and in the illustrated embodiments, each component may have different functions and capabilities other than those described, and may include additional components other than those described, which will be based on an interpretation of the scope that can be easily modified by a person skilled in the art to which the present disclosure pertains.

Therefore, the scope of the present disclosure should not be limited to the above-described embodiments, but should be determined by the claims described hereinafter as well as equivalents to the claims of the present disclosure.

What is claimed is:

1. A biodegradable circuit comprising a resistor,
wherein the resistor comprises:
a substrate;
a plurality of wirings being disposed on an upper surface of the substrate; and
a protective layer configured to cover an upper portion of the plurality of wirings;
wherein an upper surface of the protective layer is divided into:
a first region including a center of the upper surface of the protective layer; and
a second region surrounding the first region and directly adjacent to an edge of the upper surface of the protective layer, and
wherein a thickness of the protective layer in the first region is greater than a thickness of the protective layer in the second region.

2. The biodegradable circuit of claim 1,
wherein the plurality of wirings comprise magnesium (Mg).

3. A biodegradable circuit comprising a resistor,
wherein the resistor comprises:
a substrate;
a plurality of wirings disposed on an upper surface of the substrate;
a first protective layer configured to cover an upper portion of the plurality of wirings;
a second protective layer disposed on an upper surface of the first protective layer,
wherein an upper surface of the resistor is divided into:
a first region including a center of the upper surface of the resistor; and
a second region surrounding the first region and directly adjacent to an edge of the upper surface of the resistor,
wherein an area of the first protective layer corresponds to an area of the upper surface of the resistor, which is equal to a sum of an area of the first region and an area of the second region, and
wherein an area of the second protective layer is equal to the area of the first region.

4. The biodegradable circuit of claim 3,
wherein wirings included in the first region have thicker protection than wirings included in the second region.

5. The biodegradable circuit of claim 4,
wherein the plurality of wirings comprise magnesium (Mg).

6. A biodegradable electronic device configured to communicate with a wireless communication device and to control an electrical stimulator and a drug dispenser, the biodegradable electronic device comprising:
a memory unit configured to store a program including computer-executable instructions;
a controller unit configured to execute the program;
an input/output signal generator configured to generate an input voltage and an output voltage;
a biodegradable reference signal generator comprising a resistor and configured to generate a reference signal; and
a PWM (pulse width modulation) pulse controller configured to generate a signal for commanding turn-on or turn-off operations of the wireless communication device, the electrical stimulator, and the drug dispenser,
wherein the PWM pulse controller generates the signal for commanding the turn-on or turn-off operations of the wireless communication device, the electrical stimulator, and the drug dispenser based on at least one of the computer-executable instructions stored in the program executed by the controller unit, the input voltage and output voltage generated by the input/output signal generator, and the reference signal generated by the biodegradable reference signal generator, and
wherein the reference signal is adjusted based on whether or to what extent the resistor has biodegraded.

7. The biodegradable electronic device of claim 6,
wherein the resistor comprises a biodegradable protective layer and biodegradable wirings.

8. The biodegradable electronic device of claim 6,
wherein the resistor comprises:
a substrate;
a plurality of wirings being disposed on an upper surface of the substrate; and
a protective layer configured to cover an upper portion of the plurality of wirings;
wherein an upper surface of the protective layer is divided into:
a first region including a center of the upper surface of the protective layer; and
a second region surrounding the first region and directly adjacent to an edge of the upper surface of the protective layer, and
wherein a thickness of the protective layer in the first region is greater than a thickness of the protective layer in the second region.

9. The biodegradable electronic device of claim 6,
wherein the resistor comprises:
a substrate;
a plurality of wirings disposed on an upper surface of the substrate;
a first protective layer configured to cover an upper portion of the plurality of wirings;
a second protective layer disposed on an upper surface of the first protective layer,
wherein an upper surface of the resistor is divided into:
a first region including a center of the upper surface of the resistor; and
a second region surrounding the first region and directly adjacent to an edge of the upper surface of the resistor,
wherein an area of the first protective layer corresponds to an area of the upper surface of the resistor, which is equal to a sum of an area of the first region and an area of the second region, and
wherein an area of the second protective layer is equal to the area of the first region.

10. The biodegradable electronic device of claim 9,
wherein wirings included in the first region have thicker protection than wirings included in the second region.

11. The biodegradable electronic device of claim 8,
wherein the plurality of wirings comprise magnesium (Mg).

12. The biodegradable electronic device of claim 9,
wherein the plurality of wirings comprise magnesium (Mg).
